(19) United States
(12) Patent Application Publication
(10) Pub. No.: US 2006/0247443 A1
(43) Pub. Date: Nov. 2, 2006

(54) Process for making 5-methyl -N-alkyl-2-pyrrolidone from alkyl amines and alkyl levulinate esters

(76) Inventor: Leo Ernest Manzer, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/115,504

(22) Filed: Apr. 27, 2005

(51) Int. Cl. C07D 207/267

(52) U.S. Cl. 548/554

(57) ABSTRACT

This invention relates to a process for producing a reaction product comprising 5-methyl-N-alkyl-2-pyrrolidone by (a) reacting alkyl levulinate esters with alkyl amines and (b) hydrogenating the products of step (a) in the presence of a metal catalyst, which is optionally supported.

### FIELD OF INVENTION

This invention relates to a process for producing a reaction product comprising 5-methyl-N-alkyl-2-pyrrolidone by (a) reacting alkyl levulinate esters with alkyl amines and (b) hydrogenating the products of step (a) in the presence of a metal catalyst, which is optionally supported.

### BACKGROUND OF THE INVENTION

N-Alkyl-pyrrolidones can act as solvents, surfactants, dispersants, detergents and emulsifiers, and thus are useful in a wide variety of applications. N-Alkyl-pyrrolidones are components, for example, in cleaners such as industrial, metal and surface cleaners, paint strippers, printing inks, gasoline and oil additives, industrial coatings and detergents. N-Alkyl-pyrrolidones are also useful in oil and gas well maintenance, polymer synthesis, photoresist applications, agricultural and pharmaceutical manufacture and paper manufacture.

U.S. Patent Appl. No. 2005/0054861 describes a process for producing 5-methyl-N-alkyl-2-pyrrolidone by a) reacting α-angelica lactone with alkyl amines, and b) hydrogenating the products of step (a) in the presence of a metal catalyst. α-Angelica lactone has limited availability and thus does not represent a cost-effective starting material for this reaction. An efficient and low cost process for the production of diverse alkyl pyrrolidones from the more readily available alkyl levulinate esters would be desirable.

### SUMMARY OF THE INVENTION

The present invention relates to a process for making a reaction product comprising 5-methyl-N-alkyl-2-pyrrolidone. The process comprises the steps of (a) contacting at least one alkyl levulinate ester with at least one alkyl amine, optionally in the presence of an inert solvent, and (b) reacting the products of step (a) with hydrogen gas in the presence of a hydrogenation catalyst to produce 5-methyl-N-alkyl-2-pyrrolidone, wherein the alkyl groups of the at least one alkyl levulinate ester and the at least one alkyl amine are independently selected from the group consisting of —CH₃, —CH₂OH, —C₂H₅, —C₂H₄OH, straight-chain, branched or cyclic C₃ to C₂₅ alkyl, straight-chain, branched or cyclic C₃ to C₂₅ alkyl comprising at least one hydroxyl group, and straight-chain, branched or cyclic C₃ to C₂₅ alkyl comprising at least one heteroatom selected from the group consisting of O and N.

### DETAILED DESCRIPTION OF THE INVENTION

The present invention relates to a process for preparing a reaction product comprising 5-methyl-N-alkyl-2-pyrrolidone by a process that comprises the steps of (a) contacting at least one alkyl levulinate ester with at least one alkyl amine, optionally in the presence of an inert solvent, and (b) reacting the products of step (a) with hydrogen gas in the presence of a hydrogenation catalyst. Without wishing to be bound by any particular theory, the step (a) reaction is believed to produce a number of intermediates comprising one or more of those shown in the following reaction scheme, and these intermediates are believed to be converted by hydrogenation to the final product, namely the 5-methyl-N-alkyl-2-pyrrolidone.

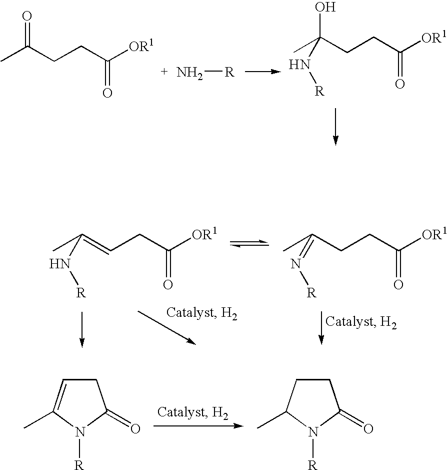

In the reaction scheme shown above, R and R¹ may be independently selected from the group consisting of —CH₃, —CH₂OH, —C₂H₅, —C₂H₄OH, straight-chain, branched or cyclic C₃ to C₂₅ alkyl, straight-chain, branched or cyclic C₃ to C₂₅ alkyl comprising at least one hydroxyl group, and straight-chain, branched or cyclic C₃ to C₂₅ alkyl comprising at least one heteroatom selected from the group consisting of O and N. The reaction may produce a mixture of products, at least one of which will be 5-methyl-N-alkyl-2-pyrrolidone.

A catalyst, with or without a support, may be present in the process of the invention to effect the hydrogenation step (b). The hydrogenation catalyst may be a metal selected from the group consisting of nickel, copper, cobalt, iron, rhodium, ruthenium, rhenium, osmium, iridium, platinum, palladium, at least one Raney® metal; compounds thereof; and combinations thereof. A catalyst support optionally may be used. A promoter such as, without limitation, tin, zinc, copper, gold, silver and combinations thereof may be used to affect the reaction, for example, by increasing activity and catalyst lifetime.

The molar ratio of the alkyl amine to the alkyl levulinate ester preferably is from about 0.1/1 to about 10/1 at the start of the reaction. Preferably, step (a) is performed at a temperature of from about 0° C. to about 150° C., and step (b) is performed at a temperature of from about 50° C. to about 250° C. In another embodiment, step (a) is performed at room temperature (about 25°). Preferably step (b) of the reaction is performed at a hydrogen pressure of from about 0.34 MPa to about 20.0 MPa. In another embodiment, step (b) of the reaction is performed at about 0.34 MPa to about 3.5 MPa.

The process of the present invention may be carried out in batch, sequential batch (i.e., a series of batch reactors) or in continuous mode in any of the equipment customarily employed for continuous processes.

In a preferred embodiment of the invention, R and R¹ are independently selected from the group consisting of —CH₃, —C₂H₅, straight-chain, branched or cyclic C₃ to C₁₂ alkyl, and straight-chain, branched or cyclic C₃ to C₁₂ alkyl comprising at least one heteroatom selected from the group consisting of O and N.

The reaction of the present invention can be performed in an inert solvent such as ethers (e.g. dioxane), straight-chain, branched or cyclic C₅ to C₃₀ alkanes (e.g. hexane), and aromatics (e.g. toluene). Preferably the reaction is carried out without a solvent.

The catalyst used in the hydrogenation step (b) may be supported or unsupported. A supported catalyst is one in which the catalytic metal is deposited on a support material by any one of a number of methods, such as spraying, soaking or physical mixing, followed by drying, calcination, and if necessary, activation through methods such as reduction or oxidation/reduction. Materials frequently used as a support are porous solids with high total surface areas (external and internal) which can provide high concentrations of active sites per unit weight of catalyst. The catalyst support may enhance the function of the catalyst.

A catalyst that is not supported on a catalyst support material is an unsupported catalyst. An unsupported catalyst may be Platinum black or a Raney® catalyst (W.R. Grace Company). Raney® catalysts have a high surface area due to selectively leaching an alloy containing the active metal(s) and a leachable metal (usually aluminum). Raney® catalysts have high activity due to the higher specific area and allow the use of lower temperatures in hydrogenation reactions. The active metals of Raney® catalysts include nickel, copper, cobalt, iron, rhodium, ruthenium, rhenium, osmium, iridium, platinum, palladium; compounds thereof; and combinations thereof.

Promoter metals may also be added to the base Raney® metals to affect selectivity and/or activity of the Raney® catalyst. Promoter metals for Raney® catalysts may be selected from transition metals from Groups IIIA through VIIIA, IB and IIB of the Periodic Table of the Elements. Examples of promoter metals include chromium, molybdenum, platinum, rhodium, ruthenium, osmium, and palladium, typically at about 2% by weight of the total metal.

The catalyst support useful herein can be any solid, inert substance including, but not limited to, oxides such as silica, alumina, titania and combinations thereof; barium sulfate; calcium carbonate; carbons; and combinations thereof. The catalyst support can be in the form of powder, granules, pellets, or the like.

In the processes of the invention, the preferred content of the metal catalyst in a supported catalyst is from about 0.1% to about 20% of the supported catalyst based on metal catalyst weight plus the support weight. A more preferred metal catalyst content range is from about 1% to about 10% of the supported catalyst. A further preferred metal catalyst content range is from about 3% to about 7% of the supported catalyst.

Combinations of catalyst and support system may include any one of the metals referred to herein with any of the supports referred to herein. Examples include palladium on carbon, palladium on calcium carbonate, palladium on barium sulfate, palladium on alumina, palladium on titania, platinum on carbon, platinum on alumina, platinum on silica, iridium on silica, iridium on carbon, iridium on alumina, rhodium on carbon, rhodium on silica, rhodium on alumina, nickel on carbon, nickel on alumina, nickel on silica, rhenium on carbon, rhenium on silica, rhenium on alumina, ruthenium on carbon, ruthenium on alumina and ruthenium on silica. Preferred combinations of catalyst and support include palladium on carbon, platinum on carbon, iridium on carbon, rhodium on carbon, ruthenium on carbon, iridium on silica, and combinations thereof.

The following examples are illustrative of the invention.

### EXAMPLES

The following abbreviations are used: ESCAT-XXX, series of catalysts provided by Engelhard Corp. (Iselin, N.J.); AWC, Acid Washed Carbon (Calsicat catalyst support from Engelhard Corp. (lot S-96-140)); SCCM, standard cubic centimeters per minute; GC-MS, gas chromatography-mass spectrometry; ° C., degrees Centigrade; g, gram; min, minute; hr, hour; ml, milliliter; m, meter; MPag, mega Pascal gauge.

For catalyst preparation a commercially available support such as carbon or silica (Grade 55; W. R. Grace, Columbia, Md.), was impregnated by incipient wetness with a metal salt. The catalyst precursors used were PdCl₂ (Alfa Aesar, Ward Hill, MA),), H₂PtCl₆ (Johnson Matthey, Inc.), RhCl₃ xH₂O (Alfa Aesar), RuCl₃.xH₂O (Aldrich Chemical Co, Milwaukee, Wis.), NiCl₂.6H₂O (Alfa Aesar) and IrCl₃.3H₂O (Alfa Aesar). The samples were dried and reduced at 300-450° C. under H₂ for 2 hours.

Ethyllevulinate, nonylamine, ethanolamine, heptylamine, cyclooctylamine and n-amylamine are available from Sigma-Aldrich (St. Louis, Mo.); Platinum black was obtained from Alfa Aesar; Carbon catalyst #69F82A was obtained from Engelhard Corp. (Iselin, N.J.).

Catalyst Preparation: 5% Pt on Calsicat Acid Washed Carbon

In a 150 ml beaker, a solution was made up of 4.5 ml 0.3 M H₂PtCl₆ with 4.0 ml deionized H₂O. To the beaker were added 4.75 g Calsicat Acid Washed Carbon (12×20 mesh, dried at 120° C. overnight). The slurry was allowed to stand at room temperature for 1 hr with occasional stirring, followed by drying at 120° C. overnight with frequent stirring (until free flowing).

In an alumina boat, in a quartz lined tube furnace, the catalyst was purged with 500 SCCM N₂ at room temperature for 15 min and then with 100 SCCM He at room temperature for 15 min. The catalyst was heated to 150° C. and held at 150° C. under He for 1 hr. At this point, 100 SCCM H₂ were added and the sample was held at 150° C. under He and H₂ for 1 hr. The temperature was increased to 300° C. and the catalyst was reduced at 300° C. under He—H₂ for 8 hrs. The H₂ was stopped, the sample was held at 300° C. under He for 30 min and then cooled to room temperature in flowing He. The catalyst was finally passivated in 1.5% O₂ in N₂ at 500 SCCM for 1 hr at room temperature and weighed 4.93 g when unloaded.

Additional catalysts used in the present invention were prepared following a similar procedure.

### EXAMPLES 1-22

### Preparation of 5-Methyl-N-Alkyl-2-Pyrrolidone

Ethyllevulinate (EtLA) and the indicated alkyl amine (R—NH₂) were mixed in approximately equal molar equivalents at room temperature (˜25° C.) to prepare a solution. To this solution was added the unsupported (Platinum black) or supported metal catalyst. The reactor was pressurized with hydrogen at the indicated pressure and heated for the indicated time. At the end of the reaction, the reactor was cooled, vented and the product analyzed by GC-MS using an HP 6890 (Agilent; Palo Alto, Calif.) equipped with a WCOT fused silica column, 25 m×0.25 MM ID, coating CP-wax 58 (FFAP)−CB DF=0.2 (Varian, Palo Alto, Calif.).

R-Pyr refers to the product 5-methyl-N-alkyl-2-pyrrolidone prepared using the indicated alkyl amine.

| | | | | H₂ | | | | | |
|---|---|---|---|---|---|---|---|---|---|
| Expt. | | | Temp | Pressure | Time | Molar Ratio | EtLA | Catalyst | R-Pyr |
| No. | Catalyst | R—NH₂ | (° C.) | (MPa) | (hours) | Ester/R—NH₂ | (g) | (g) | Yield (%) |
| 1 | Escat 142 | Nonylamine | 150 | 0.758 | 1.00 | 1.01 | 5.02 | 1.00 | 92.95 |
| | (5% Pd/C) | | | | | | | | |
| 2 | Escat 142 | Nonylamine | 225 | 0.690 | 1.00 | 1.01 | 5.02 | 1.00 | 93.24 |
| | (5% Pd/C) | | | | | | | | |
| 3 | Escat 340 | Nonylamine | 150 | 0.690 | 1.00 | 1.01 | 5.02 | 1.00 | 73.96 |
| | (5% Rh/C) | | | | | | | | |
| 4 | Escat 340 | Nonylamine | 225 | 0.690 | 1.00 | 1.01 | 5.02 | 1.00 | 78.30 |
| | (5% Rh/C) | | | | | | | | |
| 5 | Escat 440 | Nonylamine | 225 | 0.758 | 1.00 | 1.01 | 5.02 | 1.00 | 28.71 |
| | (5% Ru/C) | | | | | | | | |
| 6 | Escat 268 | Nonylamine | 150 | 0.690 | 1.00 | 1.01 | 5.02 | 1.00 | 79.91 |
| | (5% Pt/C) | | | | | | | | |
| 7 | 5% Ir/AWC | Nonylamine | 225 | 0.690 | 1.00 | 1.01 | 5.02 | 1.00 | 89.87 |
| 8 | 46.4% | Nonylamine | 225 | 0.690 | 1.00 | 1.01 | 5.02 | 1.00 | 72.46 |
| | Pt/AWC | | | | | | | | |
| 9 | Ni/AWC | Nonylamine | 225 | 0.690 | 1.00 | 1.01 | 5.02 | 1.00 | 24.52 |
| 10 | 5% | Nonylamine | 225 | 0.724 | 1.00 | 1.01 | 5.02 | 1.00 | 4.85 |
| | Re/AWC | | | | | | | | |
| 11 | 5% Ir/Silica | Nonylamine | 225 | 0.724 | 1.00 | 1.01 | 5.02 | 1.02 | 84.06 |
| 12 | Ru/Re on | Nonylamine | 225 | 0.724 | 1.00 | 1.01 | 5.02 | 1.02 | 34.63 |
| | Carbon | | | | | | | | |
| | catalyst | | | | | | | | |
| | (#69F-82A) | | | | | | | | |
| 13 | Platinum | Nonylamine | 150 | 0.690 | 1.00 | 1.01 | 5.02 | 1.03 | 75.91 |
| | black | | | | | | | | |
| 14 | Platinum | Nonylamine | 225 | 0.621 | 1.00 | 1.01 | 5.02 | 1.03 | 72.09 |
| | black | | | | | | | | |
| 15 | Escat 142 | Nonylamine | 200 | 0.621 | 1.00 | 1.01 | 5.02 | 1.07 | 11.04 |
| | (5% Pd/C) | | | | | | | | |
| 16 | Escat 142 | Nonylamine | 200 | 0.517 | 1.75 | 1.01 | 5.02 | 1.00 | 13.86 |
| | (5% Pd/C) | | | | | | | | |
| 17 | Escat 142 | Ethanolamine | 225 | 0.690 | 1.00 | 1.01 | 7.02 | 1.05 | 5.81 |
| | (5% Pd/C) | | | | | | | | |
| 18 | 5% Ir/Silica | Ethanolamine | 150 | 0.690 | 2.00 | 1.01 | 7.02 | 1.02 | 16.97 |
| | Gel | | | | | | | | |
| 19 | Escat 142 | Heptylamine | 150 | 0.621 | 1.00 | 1.01 | 5.56 | 1.01 | 95.01 |
| | (5% Pd/C) | | | | | | | | |
| 20 | Escat 142 | Ethanolamine | 150 | 0.621 | 1.00 | 1; 01 | 7.02 | 1.06 | 14.92 |
| | (5% Pd/C) | | | | | | | | |
| 21 | Escat 142 | Cyclooctylamine | 150 | 0.621 | 1.00 | 1; 01 | 5.31 | 1.13 | 93.34 |
| | (5% Pd/C) | | | | | | | | |
| 22 | Escat 142 | n-Amylamine | 150 | 0.621 | 1.00 | 1; 01 | 6.23 | 1.13 | 92.27 |
| | (5% Pd/C) | | | | | | | | |

What is claimed is:

1. A process for making a reaction product comprising 5-methyl-N-alkyl-2-pyrrolidone comprising the steps of (a) contacting at least one alkyl levulinate ester with at least one alkyl amine, optionally in the presence of an inert solvent, and (b) reacting the products of step (a) with hydrogen gas in the presence of a hydrogenation catalyst to produce the 5-methyl-N-alkyl-2-pyrrolidone, wherein the alkyl groups of the at least one alkyl levulinate ester and the at least one alkyl amine are independently selected from the group consisting of —CH₃, —CH₂OH, —C₂H₅, —C₂H₄OH, straight-chain, branched or cyclic C₃ to C₂₅ alkyl, straight-chain, branched or cyclic C₃ to C₂₅ alkyl comprising at least one hydroxyl group, and straight-chain, branched or cyclic C₃ to C₂₅ alkyl comprising at least one heteroatom selected from the group consisting of O and N.

2. The process as recited in claim 1, wherein the catalyst is selected from metals selected from the group consisting of nickel, copper, cobalt, iron, rhodium, ruthenium, rhenium, osmium, iridium, platinum, palladium, at least one Raney® metal; compounds thereof; and combinations thereof.

3. The process as recited in claim 2, wherein the catalyst is palladium or compounds thereof.

4. The process as recited in claim 2, wherein the catalyst is supported and the content of the metal in the supported metal catalyst is from 0.1% to 20% by weight.

5. The process as recited in claim 4, wherein the catalyst support is selected from the group consisting of oxides of silica, alumina, titania and combinations thereof; barium sulfate; calcium carbonate; carbons; and combinations thereof.

6. The process as recited in claim 1, wherein the catalyst is augmented with a promoter.

7. The process as recited in claim 1 wherein the process is carried out in the absence of a solvent.

8. The process as recited in claim 1, wherein the alkyl groups of the at least one alkyl levulinate ester and the at least one alkyl amine are independently selected from the group consisting of —CH₃, —C₂H₅, straight-chain, branched or cyclic C₃ to C₁₂ alkyl, and straight-chain, branched or cyclic C₃ to C₁₂ alkyl comprising at least one heteroatom selected from the group consisting of O and N.

9. The process as recited in claim 1, wherein the molar ratio of alkyl amine(s) to alkyl levulinate ester(s) is from about 0.1/1 to about 10/1 at the start of the reaction.

10. The process as recited in claim 9, wherein step (a) is performed at a temperature of from about 0° C. to about 150° C., and step (b) is performed at a temperature of from about 50° C. to about 250° C.

11. The process as recited in claim 10, wherein step (b) is performed at a hydrogen pressure of from about 0.34 MPa to about 20.0 MPa.

12. The process as recited in claim 4, wherein the supported metal catalyst is selected from the group consisting of palladium on carbon, palladium on calcium carbonate, palladium on barium sulfate, palladium on alumina, palladium on titania, platinum on carbon, platinum on alumina, platinum on silica, iridium on silica, iridium on carbon, iridium on alumina, rhodium on carbon, rhodium on silica, rhodium on alumina, nickel on carbon, nickel on alumina, nickel on silica, rhenium on carbon, rhenium on silica, rhenium on alumina, ruthenium on carbon, ruthenium on alumina and ruthenium on silica.

13. The process as recited in claim 12, wherein the supported metal catalyst is selected from the group consisting of palladium on carbon, platinum on carbon, iridium on carbon, rhodium on carbon, ruthenium on carbon, iridium on silica, and combinations thereof.

14. The process as recited in claim 1, wherein the alkyl groups of the at least one alkyl levulinate ester and the at least one alkyl amine are independently selected from the group consisting of —CH₃, —CH₂OH, —C₂H₅, —C₂H₄OH, straight-chain, branched or cyclic C₃ to C₂₅ alkyl, straight-chain, branched or cyclic C₃ to C₂₅ alkyl comprising at least one hydroxyl group, and straight-chain, branched or cyclic C₃ to C₂₅ alkyl comprising at least one heteroatom selected from the group consisting of O and N; the catalyst is supported and the supported catalyst is palladium on carbon; the temperature of step (a) of the reaction is from about 0° C. to about 150° C., the temperature of step (b) of the reaction is from about 50° C. to 250° C., and the pressure of step (b) of the reaction is from about 0.34 MPa to about 20.0 MPa.
